(19) United States
(12) Patent Application Publication
(10) Pub. No.: US 2017/0372017 A1
(43) Pub. Date: Dec. 28, 2017

(54) HEALTHCARE SELF-MANAGEMENT INTERVENTION SYSTEM AND METHOD

(71) Applicant: Casey Steffen, Oklahoma City, OK (US)

(72) Inventor: Casey Steffen, Oklahoma City, OK (US)

(21) Appl. No.: 15/194,491

(22) Filed: Jun. 27, 2016

(51) Int. Cl. G06F 19/00; A61B 5/00; G06Q 30/06

(52) U.S. Cl. CPC G06F 19/325; G06Q 30/0601; G06F 19/3431; A61B 5/4833; G06F 19/322; A61B 5/7275; G06F 19/3418

(57) ABSTRACT

A healthcare self-management intervention system and method monitors, visualizes and rewards users for participating in activities related to the self-management of a medical condition, considering channels such as diet, exercise, medication, testing, scheduling/coaching, and shopping. It is a delivery system for treatment protocols which can monitor patient engagement with said protocols through a telehealth intervention system. A digital portal may be provided for analyzing and visualizing biometric data, presenting treatment protocols and educational materials, conducting remote teleconference meetings, sending data to and from remote locations in real-time to facilitate remote appointments and grade user progress towards or away from achievement of wellness goals. The system may then award the user with digital currency associated to the positive impact to the cost curve associated to a specific disease through the positive accumulation of data that demonstrates the patient is successfully engaged in their treatment and achieving standards which lead to goal achievement. These “Wellness” points are a digital currency which can then be exchanged for cash or used for self-management supplies through a mobile shopping system.

### BACKGROUND

The costs associated with diabetes are spiraling; an increase in 41% since 2007. Currently, more than 1 in 3 adults have pre-diabetes. We now know that in 5 years' time, 5-10% of the existing pre-diabetic adult population will develop chronic type-2 diabetes. Pharmaceutical products have reached saturation levels of patient adherence leaving a majority of patients without solutions. There is however an evidence-based non-pharmaceutical solution that dramatically reduces the incidence of diabetes.

Lifestyle intervention programs that promote weight loss through dietary change and moderate intensity exercise are shown to be more effective than leading pharmaceutical products at preventing the progression of diabetes. As a result of internationally duplicated research findings, the CDC has recently implemented a national curriculum for diabetes prevention and promotes the adoption of its practices within local communities. Currently, these programs are hosted by “Lifestyle Coaches” at community facilities or hospitals. They meet weekly as a group to review patient weight loss progress and go through weekly lesson plans. Coaches prepare each patient with solutions to barriers that patients may experience in the upcoming week. Studies show these 16 week programs achieve an astounding 58% reversal rate of pre-diabetes.

According to the original study, the healthcare system has seen slow adoption of these programs. Reasons sighted include the expense to host prevention programs at clinics and healthcare facilities, difficultly to scale as a result of access to trained providers and expense of conducting. Barriers such as these require businesses to propose new solutions that create bridges between public and private sectors.

Treatment and management of chronic medical conditions continue to be one of the most serious issues facing the country. Using diabetes as an example, the American Diabetes Association estimates that there has been a 41% increase in healthcare related costs associated with diabetes from 2007-2012. As of 2012, the direct costs associated with diabetes were $245 billion, an amount that does not include lost productivity or similar costs. Similarly, the Centers for Disease Control and Prevention estimate that as of 2010, the domestic cost of cardiovascular diseases, including heart diseases and stroke, were $444 billion (again, direct costs), and accounted for $1 out of every $6 spent on healthcare.

Even in the case where a patient obtains healthcare, treatment outcomes are limited as a result of inadequate management of their treatment protocol and of the patient's health. The healthcare system faces a longstanding barrier that leads to increased healthcare expenses related to the continued progression through treatment protocols required to participate in the healthcare system. In order to address the vast majority of patients that do not reach desired treatment outcomes, preventative treatments for the progression of chronic medical conditions should be made available to the general population outside the confines of a healthcare facility which would overcome limitations that otherwise prevent access to care.

Intervention programs have been found to be useful in improving patient outcomes, but these programs are costly and are limited in the number of patients that can reliably be served. For example, some diabetes intervention programs are led by a diabetes educator and are necessarily limited in the number of patients enrolled. Other intervention programs have a particular focus that may not be applicable to all patients with a condition. For example, some intervention programs may focus on changes to diet to increase wellness, whereas other programs may focus on exercise. These limitations prevent patients in need to participate in a program relevant to their specific treatment barriers.

Patient self-care and monitoring can be useful in treating chronic conditions such as diabetes, but patients frequently fail to comply with the directions from their healthcare provider, or are prevented from receiving healthcare services due to barriers in access to care.

Accordingly, given the vast numbers of patients not receiving adequate treatment, and the resulting social and economic cost, there is thus a significant need for new strategies to help patients achieve wellness outside the confines of normally distributed care.

There is a further need for a system and method for helping patients achieve wellness, which can be made widely available to all people diagnosed with a condition and not just those receiving care from an insurance approved healthcare program.

There is a need for a system to organize lifestyle events which produce therapeutic outcomes for maximal effect throughout the entire metabolic day of the person. For instance, normal metabolic events associated to beta cell energy normalization occur after and during specific events, like rest after exercise and sleeping once glucose is normalized. To accomplish specific lifestyle synchronizations requires a system of logic and timers to notify and prompt users to take action for said maximal outcomes.

There is a further need for a system that encourages patients to financially and actively engage in positive lifestyle activities that relate to the collection of verifiable data associated to the self-management of their condition (including diet, exercise, usage of medication, bio-markers like blood glucose, HbA1c levels, weight, cholesterol, ect) which can be quantified according to the aggregate economic burden of a disease based upon the alignment of a patient's treatment vector with the ratio of patient/insurer's financial commitment to acquire that data through the purchase of supplies. This quantified treatment vector should then have actual value proportional to the financial obligation of the patient/insurer and the positive or negative offset to the aggregate burden of the disease. Any positive offset to the aggregate burden of the disease is made through the collaborative financial commitment made by patient/insurer and should therefore have a system to monitor and reward patients/insurers for successfully reducing the aggregate burden of the disease.

There is a still further need for a healthcare intervention and treatment protocol deployment system that is flexible, and can be modified to focus on a patient's specific needs present as barriers to wellness and flexible enough to suite any type of disease which requires long-term maintenance through which the system can also deliver a spectrum of disease specific treatment protocols remotely so as to provide medical training and information in order to overcome barriers associated to access of care. In ideal embodiments, the same platform for the could be used for but not limited to the treatment of obesity, chronic heart disease, hematology.

There is still the further need for a system that can reduce direct costs associated to the management of chronic conditions through the reduction in use of medication and burden placed on healthcare providers associated to increased patient demand of the healthcare system with an alternative approach to providing preventative care which modules patient engagement with lifestyle activities that produce therapeutic outcomes that would otherwise require the use of medication, or direct medical treatment only provided by the healthcare system.

There is still the further need for an exercise modulation system which can guide patients suffering from metabolic disorders to control specific biomarkers which vary in levels as a result of exercise that may otherwise produce harmful effects which hasten the progression of diseases or complications, like blood glucose becoming elevated during high intensity exercise and resulting in an increase in patient's resistance of insulin, or may otherwise produce beneficial effects that prevent the progression of said disease, like lowering blood glucose without trigger the body to raise it through the normal physiologic response associated to “fight or flight,” or stimulating the release of endogenous compounds which promote the acceleration of glucose absorption. Most significantly, for people with insulin dependent type-1 diabetes, who rely on regular exercise to reduce the complications of diabetes and also suffer from significant changes in blood glucose levels during moderate and high intensity exercise that produce life-threatening situations of hypo-glycemia or extreme hyper-glycemia depending on the intensity levels. There is a significant need for said exercise modulation system so that patients can identify when said biomarkers and patient data (heart rate, exercise levels, blood glucose, exercise duration, exercise type) that are no longer in the tolerance ranges associated to positive treatment adherent outcomes and may then trigger specific physiological response which inadvertently hasten complications, like raising a person's blood glucose or blood pressure or lowering a patient's blood glucose to dangerously low levels of hypoglycemia. Said system should guide patients through exercise activities in ways which make evident to the patient they are at risk of producing adverse outcomes, or guide them through exercise activities which keep said biomarkers within optimal ranges to produce ideal outcomes.

There is still a further need for an exercise modulation system which can reduce the daily usage of insulin stimulating medication for the purpose of reducing circulating levels of insulin within the body so that the body can return from a state of insulin resistance to insulin sensitivity. For people suffering from diabetes who experience prolonged exposure to elevated levels of blood glucose, and then require their body to produce elevated levels of insulin for prolonged periods of time, the body will become de-sensitized to the hormone insulin. As such, there is a significant need for a reproducible method of lowering a patient's blood glucose without the dependency on insulin to transport glucose from the blood into the cells. For example, a patient diagnosed with prediabetes who by definition are experiencing biomarkers associated to metabolic decline and some degree insulin resistance should have access to an exercise modulation system which can reduce their dependency on prescribed insulin-stimulating medication, or insulin itself so as to reduce insulin resistance and allow a preventable metabolic disorder to return to a state of metabolic health. For many people who fail to utilize exercise effectively for the purposes for losing weight and preventing diabetes may in fact make a preventable medical condition worse and lock them in a state of permanent insulin resistance as a result of raising blood glucose as a result of the “flight or fight” response associated to heart rate and exercise levels or have no effect exercise modulation system which can guide a patient reduce their daily insulin demand.

There is a further significant need for an exercise modulation system which can reduce the daily usage of insulin based medication for the purpose of reducing circulating levels of insulin within the body so as to reduce the financial obligation the patient and insurer share in the long-term maintenances of chronic medical conditions through the increase demand in medication. Patients should have an effective exercise modulation system which can effectively reverse the progression of a preventable disease, or improve outcomes in the treatment of said diseease, so as to reduce the financial and emotional obligation associated to managing said condition and then needlessly depending on an excess of medication to manage a preventable condition.

There is a further need for an exercise modulation system which can acutely lower elevated blood glucose so as to maximize weight loss associated to moderate intensity exercise within target heart rate ranges for specific lengths of time. In order to replenish stores of glucose reserved for immediate access to energy to continue exercise activity, the body will either trigger the fight or flight response depending on different triggers, like heart rate, and raise temporarily raise blood glucose to make energy available to the muscle. But if an exercise modulation system identifies these triggers, and alerts the user to change exercise levels before triggering the fight or flight response or reduce exercise levels too much as to not trigger fat cells to release their stores of glucose. Therefore, in order in order to achieve therapeutic treatment outcomes, people suffering from obesity, heart disease, metabolic disorders like diabetes, must first achieve adequate levels of blood glucose before the body releases its fat stores, and as a result of this requirement, there is a significant need for an exercise modulation system which modulates people engagement with the exercise activity so as to achieve treatment outcomes related to weight loss.

There is still the further need for an exercise modulation system which can acutely lower elevated blood glucose so as that people suffering from type-1 diabetes can reduce the risks associated to type-1 diabetes and reduce the use of insulin due to the life threatening risks associated to extreme hypoglycemia resulting from exercise, or extreme hypoglycemia resulting from exercise. Currently, there is no system which enumerates these ranges (heart rate ranges, duration of exercise, exercise pace and level of exertion, exercise-induced glucose absorption rate associated to a specific exercise type and proximity to insulin dose, starting blood glucose, time to muscle glucose store depletion, blood glucose recovery period post exercise, and number of data point samples to determine exercise-induced glucose absorption rate) and tolerances for these various exercise levels so as to prevent adverse healthcare complications.

There is a significant need for a system which can modulate various factors associated to engagement with exercise activities which maintain a patient's heart rate within ideal ranges while exercising for specific durations of time at said exercise levels, without triggering the “flight or fight” response which would then releases glucose from the liver in an attempt to replenish the stores of glucose in muscles and then raise blood glucose, so that instead they can then safely exercise in a way that will allow them to lower blood glucose levels without the use of medication, re-sensitizing the a body suffering from insulin-resistance.

There is still a significant need for an exercise modulation system which can specify a predetermined exercise period so that users are alerted to the length of time to continue to exercise at said exercise intensity so that the user prevents adverse exercise outcomes.

There is still an even greater need for a system which can operate remotely, be administered by healthcare professionals, collect data associated to treatment engagement, submit that data in an electronic format which can be verified for its authenticity in order to re-reimburse both provider and patient for their successful engagement with various health services which produced improved patient outcomes.

### BRIEF DESCRIPTION OF THE APPENDIX

The features and advantages of the present disclosure will be more fully understood with reference to the drawings and figures included as Appendix A.

### DETAILED DESCRIPTION

System runs measurements associated to multiple different types of biometric data which define a series of system parameters and interpreted by rules which define visualizations of data either, numerically or graphically, and monitor that data stream for trends based on a set number of previous data entries in order to determine if and what type of action is required to correct or accelerate progression towards treatment goals.

As will be discussed in greater detail below, in embodiments, a computing device is provided for managing a user's interaction with the system. Computing device 110 may be fixed or mobile and provides a system display in the form of a digital portal 102 tracking patient progress towards wellness goals, deviations from ideal treatment vectors, and provides access to educational training related to self-management of their condition, and guides them through safe and treatment adherent exercise activities.

In embodiments, a digital portal 102 may be configured to receive biometric information about the user's condition and alert the patient and healthcare provider to trends in data progression, modulate current exercise levels and duration to achieve ideal treatment goals, a flexible portal through which to relay different treatment protocols, medical education, denote user performance, projected date for goal completion and instructions for better management of certain wellness activities, as well as incentives to properly manage care and promote engagement with said activities. In embodiments, a system dashboard provides users with a heads-up display of the total metabolic system performance through the use of various graphic shapes and elements which represents values of various measurements which describe the major factors that impact person's metabolism.

Computing device 110 may be any type of device capable of running application software. For example, computing device may include smartphones. (e.g., Apple iPhone, Apple Watch, smart watches, or devices running the Google Android OS, devices running the Windows Mobile OS, or any other variety of operating system and digital platform such as personal digital assistants (PDAs), tablet computers and touchscreen tablets (e.g., Apple iTouch, Apple iPad, Barnes and Noble Nook, Amazon Kindle Fire), e-Readers, media players, and/or other handheld computing devices.

Computing device 110 may also take the form of a personal computer such as a desktop or laptop computer running an operating system (e.g., Microsoft Windows, Max OS X, Linux, etc.) and having the capability to run application software or load data through a web portal which replicates the system functionality. In alternate embodiments, computing device 110 may be a remote server that serves data to a client computer or display over a network such as the Internet. An example of one such configuration is an HTTP-based client-server system or a virtual personal network or VPN. In alternate embodiments, the underlying architecture and theories which define a digital embodiment may also be applied to the delivery of specific services.

In embodiments, a digital portal 102 may be a digital projection of the data captured by the system and arranged in manner to sufficiently communicate the interconnected nature of metabolic measurements visually. In this interaction of the Digital Portal, it shall be referred to as a mobile app that is designed to run on the particular class of computing device. For example, an app specific to the iOS operating system could be made available via the Apple App Store to iPhone users. In embodiments, a version of digital portal 102 could be made available to Google Android users through the Google Play store. In alternate embodiments, digital portal 102 may incorporate so-called smart televisions, connected televisions, or Internet-connected televisions. In embodiments, digital portal 102 may incorporate digital media devices such as Apple TV, Google Chromecast, Roku, TiVo, or the like. In embodiments, the digital portal may be deployed through web-browser. Versions of a digital portal app or application 102 could be made available for all relevant operating systems and devices.

In embodiments, digital portal 102 can be software that runs on a remote server and which is accessed through a communications network (e.g., the Internet) using HTTP, VPN, or any other network-based communication system and displayed remotely.

The specific features of digital portal 102 will be discussed in further detail below.

Data collection from biometric sensor's, dietary information, and medication usage are a vital part of this system. Data collection is facilitiated by allowing manual input of data or by automatic detection through a variety of computer aided communication protocols including Bluetooth, Wifi, or radio frequency. Accessing the EIGAR system is accessed through the Add Activity pop-up boxes and uses the automatic detection protocols mentioned above.

In embodiments, a biometric sensor 120 may collect physiological information about the patient's condition for further analysis or evaluation by the system. In embodiments, biometric sensor 120 may be a passive blood monitoring device, at-home glucose monitor, contact lens sensor, sports monitoring sensor, pedometer, heart rate monitor, blood pressure sensor, scale, thermometer, or any type of device that collects information relevant to the patient's condition for use by the system of the present invention.

In embodiments, an off-the-shelf solution may be incorporated into the system of the present invention. For example, TomTom, Polar, Nike, and other companies manufacture consumer-grade sensors that aggregate data such as dietary data, body weight, heart rate, cadence, and distance traveled, and these types of components can be incorporated without departing from the scope of the present invention.

In embodiments, sensor 120 might be a scanner to read codes on prepared meal of known dietary values. For example, Nutrisystem and Jenny Craig pre-package calorie-reducing meals, raw ingredients to prepare meals of known dietary value packaged into containers with said sensors and these types of prepared meals can be incorporated without departing from the scope of the present invention.

In embodiments, biometric sensor 120 may be a pressure, motion, or weight sensor that calculates whether the patient has taken a prescribed medication and the quantity of each dose. In embodiments, a patient's medication consumption may be monitored by measuring changes in the weight of the prescription container. In embodiments, a pressure sensor may be utilized to confirm that a medication container has been lifted off the sensor. In still further embodiments, sensors may be used to determine changes in a syringe plunger or syringe pen to verify that a patient has taken a syringe-based medication and the quantity of each dose.

In embodiments, a connection 130 provides a link between sensor 120 and computing device 110, and may be by any known means that will permit the reliable transmission of data between computing device 110 and biometric sensor 120. In a preferred embodiment connection 130 is a wireless connection such as Bluetooth, WiFi, radio frequency (RF), ultra-high frequency (UHF), or the like.

In embodiments, connection 130 can be a data cable such as universal serial bus (USB), Toslink, Ethernet, Lightning®, Thunderbolt®, HDMI or the like. In still further embodiments, connection 130 can be accomplished using storage media such as an SD card, flash memory drive, CD-ROM, or any other storage medium the is coupled or inserted into computing device 110 or biometric sensor 120, loaded with data, and then manually transferred to the corresponding device.

In other embodiments, connection 130 may be accomplished via a network connection such as the Internet, corporate network, VPN, or the like. In other embodiments, connection 130 may involve manual data entry wherein the user takes data from biometric sensor 120 and manually inputs that data into computing device 110, or vice-versa.

In embodiments, data crossing connection 130 stream between sensor 120 and computing device 110 may be encrypted to protect patient confidentiality.

In embodiments, computing device 110 may connect to a remote healthcare server 140 that collects information from the user for analysis by healthcare professionals.

In embodiments, a remote healthcare server 140 may be provided to store patient data or allow access by healthcare providers. In embodiments, server 140 may be connected to computing device 110 to receive data concerning the patient's condition.

Server 140 may take the form of any server known in the art that permits secure storage and retrieval of data. In embodiments, server 140 may be dedicated device running a server operating system such as OSX Server, Windows Server, or a Linux-based system such as Ubuntu. In embodiments, server 104 may be a cloud-based system such as Google Documents, Box, DropBox, or the like. In embodiments, a hosted server solution may be used such as Rackspace, Amazon, EC2, or similar.

In still further embodiments, an e-mail server or SMS gateway may be utilized to communicate information to healthcare providers, or to communicate information to those healthcare providers from the patient. For example, in embodiments, healthcare providers could elect to receive e-mails, SMS messages or other notifications concerning patients.

In embodiments, server 140 may be omitted, and the healthcare provider may interact directly with the system. For example, in embodiments, a healthcare provider directly accesses computing device 110 for analysis.

In embodiments, the data stored on server is encrypted or stored in a manner consistent with governmental or industry privacy standards such as The Health Insurance Portability and Accountability Act (HIPAA).

In embodiments, healthcare providers may be enabled access to server 140 to review or analyze information concerning the patient, and provide recommendations, information, or other feedback. Such access may be by any means known in the art such as a virtual private network, web-based portal, client-based software, mobile app, or the like.

As discussed above, the present invention is applicable for use with all types of data transmission or communication networks and computing platforms. For the sake of simplicity, discussions will concentrate mainly on a mobile application running on a mobile device in communication with a remote server, via a communication network, although the scope of the present invention is not limited thereto.

In exemplary embodiments, the system is composed of a digital portal 102 that provides a system display tracking user progress towards wellness goals and provides access to educational training related to self-management of their condition.

In exemplary embodiments, the system may also draw data from other types of sensors related to the prevention of diabetes related complications or treatment protocols. Such data may include measurement for the presence of Albumin screening in urine, eGFR, neuropathy screening, and insulin usage.

Local HIPAA Compliant Data Storage and Communication as embodied by Apple Health Integration

Apple Health is a data storage and HIPAA compliant communication system. Much of the data written to Apple Health by other 3rd party apps is of use to visualizing metabolic data. By reading and writing data to Apple Health, the MDIS App provides people engaged with diabetes prevention programs a unique opportunity to work closely with Lifestyle Coaches to review data in a modern, realtime manner.

One of the App's most important functions is deployment of treatment protocols and educational programs remotely through a deployment architecture which can be configured to any disease or treatment. Curriculums like the CDC's Diabetes Prevention Program, or custom treatment protocols associated to other diseases, are hosted via remote servers and websites. Weekly and daily activities associated to the long-term care and engagement with treatment are added to the scheduling system. Each of these activities is then fully described through various windows and pages of the app. To adhere to the needs of specific healthcare providers and treatment plans, the data collected directly by the system can then be exported out in various data type formats, including EMRs. (this should adhere across any mobile platforms not just iOS and Apple)

Remote and Local Modulation of Patient Engagement with Treatment Activity in Real-time

A digital portal which can monitor, organize, transmit, capture, analyze and react to data associated patient engagement with a treatment protocol activity in real-time by modulating patient performance of said activity is a necessary and novel solution for the prevention of medical conditions. The Digital Portal and underlying system provides the necessary pathway for patients without sufficient knowledge or cognitive presence to remember how to achieve treatment goals be reminded about, or guided to treatment outcomes.

### Digital Portal

The Digital Portal is to be known as the visualization of data the system stores which defines the various interconnected aspects of patient engagement with the treatment protocols. Referring to the figures, an overview is shown of an exemplary digital portal in the form of a mobile app according to embodiments of the invention. Channels for are provided for exemplary categories self-management and may include, for example, diet, exercise, medication, testing, scheduling, and shopping. The channels provided may vary according to the particular type of condition being treated.

The digital portal enables patients to access wellness programs outside the confines of existing medical facilities by using digital consumer products and remote EMR data communications to monitor and modulate patient engagement with the prevention program while helping patients understand their progress towards treatment goals.

In embodiments, the digital portal is designed to support patients in their goal of losing weight by removing barriers associated to accessing healthcare treatment. It captures, visualizes, securely records, and analyses patient data for looking for trends in performance associated to the self-management of chronic medical conditions like diabetes. The digital portal is designed to be simple to communicate metabolic information intuitively to a wide variety of patients and healthcare professionals.

### Measurements, Activities, Goals, Target Ranges, Actions, and Data Trend Analysis

A component of the system is the measurement of biometric and lifestyle data associated to the maintenance of a chronic condition collected on a daily basis during Activities related to their treatment protocol. Each Activity has a Goal Value. Each Goal Value has a Target range which defines a threshold for values that still fall in an ideal zone and trigger Actions, while also identifying whether a new data entry is moving towards, away from a goal value, or it's undetermined. Actions are a variety of different responses to trends over the course of a period of time, such as, pop-ups, emails, emergency communications, scheduling activities or playing recorded media. Those responses are only limited to mobile phone actions, but may include actions outside of the framework of the system. Trends in data are associated to continuous data entries in-range or out-of-range values. Target Ranges are further modulated according to the during of the activity related to the data entry. For example, some settings may define a value/day but an activity slot may be in terms of the duration of the activity. The Target Range for that data type is then resized according to dashboard requirements. When various ranges of activity data are input, the system compares that data to trending data in order to determine if an action is required, or need be flagged for the future as out-of-range. For other types of notifications, after a specific amount of data points within various ranges occur, then different that type of notification would be triggered. For example, when data entered achieves target goals, like weight, then an action would immediately be sent for the goal completion. For other notifications like trending away from a goal, data entered over the course of multiple entries which continue to as out-of-range values would trigger an action when a specific amount of entries of that data type labeled with that trend is reached. The amount of entries which would trigger an action can either be user defined or treatment defined.

### Dashboard and Metabolic Graph: Overview

Referring to the figures, an interactive metabolic graph is shown. In embodiments, the interactive metabolic graph may be a visualization tool used to align different types of measurements by date and time and normalized in height according to treatment goals such that the middle of the graph represents the target value for each measurement. This target goal is coordinated about a target zone (darker blue bar in the middle of the graph) defined by user specific goals. Selecting each shape on the metabolic graph will provide users access to more layers of data collected during measurement and activity. The Digital Portal will load the information into informational boxes at the top of the graph. Using various navigation techniques, the user can load alternative days into the graph for review as a method to review data over a period of time.

Below the interactive graph may be colored boxes that allow the user to input new data for each category (“+”) and review current information about their status (Active time total of “50 minutes/week”) or point totals. Users can select different data attributes by clicking on the colored radial buttons below the colored rectangular boxes. In alternate embodiments, users can add or remove radial buttons and data types tracked by the system, use of alternate shapes and graphics to display data according to different modalities. In ideal embodiments, the user can zoom into the metabolic graph for each data point and reveal the data visualized with a different modality. In the case of heart rate data, in ideal embodiments, the user is able to zoom into the graph and the vertical bar, which denotes either the Min/Max heart rate or Average recorded heart rate, would be displayed as a curvy-linear line showing the modulation of in heart rate over time. Or, in the case of “steps”, zooming into this data may reveal steps not as bar but rather a curvy-linear graph.

The Dashboard is visual organization of data and analytics associated to representing a user's data profile as it relates to a chronic medical condition and allows for the visualization of a user's progress in their treatment protocols. As such, the dashboard should be able to be configured into a variety of arrangements suited to both digital platform for display, user specific measurements which define categories of focus required by their treatments protocols. As such, display of measurements (calorie, heart rate, glucose, medication, blood pressure, etc.) should be able to be added or removed depending upon the data type and medical condition of interest. For instance, in application of the system to alternate medical conditions like heart disease, rather than monitoring blood glucose, the user may instead want to track blood pressure, or may choose to eliminate tracking weight as a measurement entirely, removing it from display on the dashboard.

By clicking on the “+” button, a dialog box will appear that prompts the user to input data manually, or capture it automatically through electronic communication, or invoke the EAGAR system capture. This dialog box will allow the user to select the date and time of the activity, and enter salient information about that event, categorizing it by time of day, activity type or any other situational parameters which have no automatic detection for said parameters.

An important function of the app is to automatically track the time the user spends engaged in exercise activity. In embodiments, the digital portal may interface with health tracking sensors and monitors such as smartphone, smart watch, diagnostic tests conducted outside the requirements of a medical condition, or external pedometers. These sources of data are then analyzed and combined with other parameters to define necessary parameters for the operation of said system. In the instance of monitoring a user while walking, the amount of steps taken over incremental periods of time defines the Pace for the activity and used by the EIGAR system as the input for which is then monitored and triggers display changes and notifications. Monitoring for data points whose values exceed target ranges is an integral operation performed in real-time, in the instance of walking, counting the total time spent above those target “steps/minute” range will add increments of Active Time and counted towards the patient's specific Active Minutes/week goal. In the instance of other exercise activates like swimming, Pace may alternatively be defined as “Strokes/min.”

### Activities: Overview

The Activities page is a catalog of weekly treatment adherent activates tailored to the needs of individual patients and their interests. These activities may include but not be limited to, including treatment curriculum, exercise directions and examples, meal and recipe ideas, shopping, and testing various biometrics. Many people find it difficult to find time for these activities in their daily lives. A direct requirement of this system is the functionality to schedule these activities and insert them into the user's digital calendar.

In ideal embodiment, activities conducting remote person-person meetings through the use of a smartphone or computer and allow the user to submit digital responses and fill out digital forms to engage with curriculum associated to that meeting or treatment protocol.

User can select each activity and chose to either delete, schedule or read more about that specific treatment adherent lifestyle activity. By deleting the activity, the user is allowed to provide preferential information about lifestyle activities suited to them. Scheduling the activity will add it to the user's mobile phone calendar, triggering notifications to alert the user to the coming activity. By clicking on Read-more, alternate pages and windows will allow the user to more fully engage with that content to gain knowledge, make purchases, fill out forms, communicate with coaches and healthcare providers, watch videos, and other necessary or useful types of visualizations or system functions.

Activities description and information are stored remotely, as a database or webpage, and accessed by the system remotely so as to allow for rapid customization according to the needs of the user.

### Exercise-Induced Glucose Absorption (EIGAR) Exercise Modulation System

A significant physiological response happens during exercise which would otherwise require the use of insulin-based medications to achieve, acute lowering of blood glucose. For people with insulin-dependent diabetes, this poses a significant risk allowing patients to dangerously lower blood glucose during exercise. At the same time, if people with insulin-dependent type-2 diabetes exercise at intensities too elevated for too long a period of time, the body will naturally raise its blood glucose, potentially to dangerously high levels. For others who suffer various types of metabolic disorders related to elevated blood glucose levels and insulin sensitivity, exercise is a vital component of necessary activities which prevent healthcare disorders. There is a significant need for an exercise modulation system which can both lower blood glucose to ideal ranges, and increase insulin sensitivity related to exercise.

The EIGAR modulation system is based on the continued normalization and comparison of data related to the changes in blood glucose, heart rate, pace, exercise intensity and duration. The system stores rates of glucose absorption calculated from pre and post exercise blood glucose score and stores that data in conjunction with by the exercise type, duration, intensity and if the person had taken insulin within the last hour. The EIGAR Delta is the changes in blood glucose resulting from an exercise activity and the time spent in target heart rate zones during the exercise activity. The EIGAR Rate is the average glucose absorption for a specific exercise type over the course of 1 minute at target heart rate. The EIGAR Score is an average of a specific number of preceding EIGAR Rates which is then applied for use in real-time modulation of an exercise activity. The EIGAR Counter is used to estimate the pace at which glucose is absorbed by muscles during exercise. The EIGAR Counter is initially set by the user at the start of the exercise session by checking the user's blood glucose score and recording it in the system. The EIGAR Counter decreases or increases at the pace set by the current EIGAR Rate every minute of exercise so long as the user is exercising at or above target heart rate zones and intensities. As the EIGAR Counter approaches target blood glucose scores or out of range values, the system alerts the user to the trend and directs the user to stop exercising, check blood glucose, stop or reset the EIGAR Counter to most recent glucose score.

In order to trigger the Active Time Counter, two biometric parameters must and the EIGAR Rate must be above target goal minimums. The requirements for reaching these minimums are directly related to the user maintaining heart rate, and exercise intensity levels for long enough so that muscles begin to deplete their stores of energy and begin extracting a specific rate of glucose from the blood stream. Leaving these target ranges will triggers notifications which alter the user to change behavior and either slow down, speed up, pause and re-test blood glucose, or stop exercising.

In ideal embodiments, the EIGAR system will monitor carbon dioxide output from the lungs and compare the difference between resting rates of CO2 output with CO2 output during exercise to determine the amount of glucose burned through either Glucose Absorption or fat burning.

### Data: Overview

The Data page is offers a user insight into data collected by the system based on viewing entries chronologically and or filtered by data type. The Data page provides trend reports for users to understand subtle information associated to their behavior which would otherwise be problematic, preventative and cumbersome to do by hand. Trend reporting analyses measurement data by type and aggregates and organizes this data not by time, but by activity type and value range of each data entry. One type of trend reporting for the system is to display the percentage of above target range data, below target range data, or inside of target range data for a specific type ID of data. For example, understanding how often a person has elevated blood glucose waking in the morning is an important visualization which communicates the tendency of the user to stay within ideal ranges for the majority of data collected.

### Settings: Overview

In embodiments, a user is able to modify various system settings to modify how the digital portal and trend monitoring system operates and displays information. By engaging the Side Menu button or bottom button (three horizontal bars at the top left of the screen or fourth button from the right on bottom). The Settings page controls which data categories (Diet, Exercise, Medication, Blood, and Weight) are displayed, displays controls for actions and notifications overrides, point allocations for activities, displays remaining works app will remain functional before cycle ends, and presets which define a generalized system settings for patients of specific disease types. For instance for a person with pre-diabetes, the category for medication may be turned off by default since these individuals may not be receiving medication yet to treat their metabolic disorder. Or, some users may not wish to track and display certain activity on the Dashboard, and turn off the setting to do so. Additionally, attributes of each category can be turned on or off based on user interest. The attribute “Protein” is turned off in the “Diet” category and not shown in the Interactive Graph displayed on the Dashboard. This should remove that data from the metabolic graph in addition to under the colored bars. Other features include:

a. Controlling what information is communicated to share with the smartphone and their healthcare providers is in the hands of the user which can be toggled on and off to disable that information exchange.

b. When users reach or fail to goals, the trend monitoring system recognizes these events and triggers the system to initiate a variety of actions. These actions range from pop-up windows on their phone that let the user know of their success and award them with additional points or notification emails can sent to healthcare provider if they fail to accomplish goals like logging daily calorie intake. This functionality can be turned on and off and modulated by the user based on their interest or needs in the settings page.

c. Points are awarded to users for successful engagement with the treatment protocols monitored by the system. The allocation of these points for the successful degree of engagement with the treatment adherent activity is defined in the settings page. For instance, on Monday morning, points in each category are reset to zero. Each week, points are totaled and reported in weekly PDF export. That week's point total will be compared to a High Score, and included in the report. The settings page will allow the user to enable or disable point calculation, BUT NOT change the rate of point awarded.

In ideal embodiments, points values are defined based upon a wide demographic of user performance data which normalizes success for each person in negative offset to the cost curve associated to the disease created from successful biometric results captured by the system, attributing equal values for each user not only for successfully entering data, but rather a weighted value of points associated to successfully entering data, and the

In embodiments, users may be awarded points for:

Successful data input=1 point. 2. Data input falls into target range=1 point. 3. Data input reaches goal=5 points. If 1 point, created by adding new data, is associated to cost reducing gains of engagement with a treatment protocol, then that 1 point scaled accordingly in order to normalize.

Trial Period defines the weeks remaining of the app functioning on the user's phone. The trial period is defined as the length of time for the treatment protocol to last, in ideal embodiments, at the end of which, the user need to re-subscribe.

Settings include measurements, counters, sample periods, activities, conditions, rules, and actions.

Each measurement category (Diet, Exercise, Medication, Blood Testing, Weight) has a top level of system settings which can be easily accessed via the first page of the settings. For in-depth definition of system wide parameters which are associated to the operation of the system are defined in Category specific settings pages. Each Category Settings topics include: Measurement parameters, Counters, Sample Periods for data aggregation, Activities, Conditions, Rules and Actions.

Measurement: Measurements of user engagement with treatment protocols is a comparison of data captured from a variety of sources which is then compared to a target goal associated to treatment outcomes. Components include:

(a) Name/Type: the type of measurement being collected. This may range and vary depending on the type of disease or healthcare issue being monitored.

(b) Conditional: Determines how the goal should be measured

(c) Goal Value: The target value for each measurement

(d) Unit: Determines the unit of measurement depending on the devices or region of the world the user is located

(e) Run-time/every: The frequency by which the measurement is made. (end of day, every hour, on user input)

(f) Add Run Time: Adds an addition measurement based on user's interest.

Counter: The counter is the link between remote data and local installation of the system. The counter loads relevant data and assigns them to working variables of the system. Variables may include but not limited to:

(g) Data Source: Is this data stored locally on the phone in Apple Health, or in internal memory. Is the data source remote on servers? These settings can be defined here

(h) Current Value, Goal Min, Goal Max: Current value of the most recent data entered, Current data point's minimum and maximum target range value

(i) Previous Value, Goal Min, Goal Max: Current value of the most recent data entered, Current data point's minimum and maximum target range value

(j) Total Average, Minimum, and Maximum Value: The average value for each data entry over the course of the current data sample period for the all data entry points, and the total maximum and minimum data point entered.

(k) Points Awarded: Total points awarded for the specific measurement type.

(l) Points Possible: Total number of possible points for the specific measurement type.

(m) Counter Samples: Number of data points included in the counter for analysis.

Activities: Activities are defined by various treatment protocol parameters. Activities are lifestyle behavior events which can be measured by our system and customized to the user's interests. Examples may include: Lunch, Fast Blood Glucose, Going for a Walk, or measuring a person's weight. Activities can be added to the system by the user so as to facilitate customization or their specific lifestyle barriers. Activities have the following properties, but not necessarily limited to:

(b) Activity Name: Type of activity being measured

(c) ID: Unique identifier so that a single activity type can have multiple instances

(d) Time: Time is denoted as either based on hours of the day, or duration of time

(e) Day: Frequency of measurement. Some measurements are taken on a weekly basis, some everyday.

(f) Goal Target: Goal for the entered data for the user's activity

(g) Goal Min: Offset for the minimum value to be accepted as within range.

(h) Goal Max: Offset for the maximum value to be accepted as within range.

(i) Exercise activities have a unique attribute associated to triggering Active time. This Pace trigger by which the EIGAR system begins its initiation process. Examples of triggers include: Steps/minute, strokes/minute, spins/minute, reps/minute, or time spent above a certain heart rate.

Conditions: Conditions determine the logic behind the trend analysis of the data. Conditions load variables from the counter and compare them to other values in the data set. Conditions allow the user, or development team, to customize data analysis to the needs of the user or type of data being accessed. Conditions have a parameter, a goal and an operator to compare the parameter to the goal.

Rules: Rules trigger actions based on conditions being satisfied. Rules are If-then logic statements such that if conditions are met or not, the result is either action or no action.

Actions: Actions are returned when rules are met. Actions require 3 basic parameters in order to execute: Activity action to access, Notification type to use, type of trend identified. Actions may include any variety of digital communications depending on the digital portal type and not necessarily limited to sending emails, pop-up windows, in-range and out of range values, and notification for goals reached.

### User Profile: Overview

Referring to the figures, a user profile entry system is shown. The User Profile stores basic information about the user for the program to access. Coaches should work with users to make certain their User Profile is set up correctly to ensure the App functions and displays information correctly. The User Profile also stores contact information for important people, such as the user's coach, or emergency contact. The User Profile stores any patient specific data required to initiate the system, such as system start date or body mass index (BMI).

Referring to the figures, a home screen of an exemplary digital portal 102 is shown, which displays the user's progression towards achieving self-management goals. In embodiments, each category displays the user's current accumulation of “wellness” points for that week. These points are deposited when reset and used in exchange for products and services of comparable value. This value is to be draw in part from re-imbursements to the user and payments made to the healthcare facility for reaching performance standards and goals.

The digital portal is designed to help users reach wellness goals by communicating their progression towards or away from their goals. Goals are initially set during the initial user registration process and can be changed via the User Profile, located in the side menu. To change user goals, click the menu button on the top left (three horizontal stacked bars) and then click the User Profile button.

The figures disclose the dashboard of a digital portal 102 according to embodiments of the invention. Each category dashboard provides a heads-up display of recent biometrics and goal achievements. In embodiments where exercise is a criteria, the display can visualizes a patient's recent exercise performance providing a visual guide understanding how little or much they have exercised.

In embodiments, additional pages of the app may be included within the digital portal 102. These pages may include an activities description page, and an activities scheduling system which assigns different treatment activities to different days and times. In embodiments, there could be additional pages which include text messaging and video conferencing functionality vital for the usage of the app to conduct remote patient-provider appointments. In ideal embodiments, the app includes a points exchange/shopping page which allows users to exchange points for rewards. Rewards can range from any type of monetary compensation of proportional value to the verified data within treatment protocol target ranges.

The figures disclose elements of a diet page of a digital portal 102 according to embodiments of the invention. Features of the diet category include tools to empower patients with choices to make smart decisions when it comes to diet.

The figures disclose additional elements of the digital portal 102 according to embodiments of the invention. In embodiments, the user may be presented with localized on-the-spot solutions at restaurants, grocery stores, walking trails, or group meetups with smart suggestions that help users achieve treatment goals.

The figures disclose still further elements of the diet section of a digital portal 102 according to embodiments of the invention. For some users, weight loss is a vital element to their self-management treatment. Defining ideal caloric intake through prepared meals ensures these at-risk patients, and those whose medical issues requires weight loss, can subscribe to a meal preparation service to have meals and fresh produce delivered or available for pickup at local facilities. In embodiments, food delivery services can be incorporated to insure availability to at-risk low-income neighborhoods without local access to fresh produce.

Since adherence to a medication schedule is a vital component to self-management of certain conditions, remembering daily dosages can be difficult, and verifying that patients take medication is important to activities to engage in during lifestyle intervention. In embodiments, sensor driven information connecting prescription containers (syringes, vials, bottles) to the system will provide verifiable data about real-time medication usage without requiring the patient to input data.

The system of the present invention is not limited to healthcare applications and can be used in any environment where positive reinforcement of a participant's participation in certain behaviors is desired. For example, the system of the present invention could be used to encourage a student to study, do their homework, and partake in other educational activities, and to reward the student for their participation. Alternatively, the system of the present invention could be used to encourage employee behavior in certain categories.

It will be understood that there are numerous modifications of the illustrated embodiments described above which will be readily apparent to one skilled in the art, such as many variations and modifications of the compression connector assembly and/or its components including combinations of features disclosed herein that are individually disclosed or claimed herein, explicitly including additional combinations of such features, or alternatively other types of contact array connectors. Also, there are many possible variations in the materials and configurations, graphical displays and compositions. These modifications and/or combinations fall within the art to which this invention relates and are intended to be within the scope of the claims, which follow. It is noted, as is conventional, the use of a singular element in a claim is intended to cover one or more of such an element.

Detailed embodiments of the disclosure are disclosed herein; however, it is to be understood that the disclosed embodiments are merely exemplary and may be embodied in various forms. Therefore, specific details disclosed herein are not to be interpreted as limiting, but merely as a basis for the claims and as a representative basis for teaching one skilled in the art to variously employ the disclosure in virtually any appropriate manner, including employing various features disclosed herein in combinations that might not be explicitly disclosed herein.

Further, while the disclosure utilizes diabetes as an illustrative medical condition that can be treated using the present invention, this use is intended only to be exemplary, and the present invention can be used to treat any patient condition that is amenable to at least partial self-management of lifestyle factors, including cardiovascular disease, obesity, hypertension, and others.

### Export: Overview

In embodiments, a user is able to modify various system settings to modify how the digital portal and trend monitoring system operates and displays information. By engaging the Side Menu button or bottom button (3 horizontal bars at the top left of the screen or fourth button from the right on bottom). The Settings page controls which data categories (Diet, Exercise, Medication, Blood, and Weight) are displayed, displays controls for actions and notifications overrides, point allocations for activities, displays remaining works app will remain functional before cycle ends, and presets which define a generalized system settings for patients of specific disease types. For instance for a person with pre-diabetes, the category for medication may be turned off by default since these individuals may not be receiving medication yet to treat their metabolic disorder. Or, some users may not wish to track and display certain activity on the Dashboard, and turn off the setting to do so. Additionally, attributes of each category can be turned on or off based on user interest. The attribute “Protein” is turned off in the “Diet” category and not shown in the Interactive Graph displayed on the Dashboard. This should remove that data from the metabolic graph in addition to under the colored bars.

It will be understood that there are numerous modifications of the illustrated embodiments described above which will be readily apparent to one skilled in the art. These modifications and/or combinations fall within the art to which this invention relates and are intended to be within the scope of the claims, which follow. It is noted, as is conventional, the use of a singular element in a claim is intended to cover one or more of such an element.

We claim:

1. A method for healthcare self-management intervention comprising:
generating on a server computing a treatment protocol for a user;
receiving from a digital portal running on a portable computing device at least one metric concerning a user's health and/or activity;
processing said at least one metric to track the user's engagement with the treatment protocol;
grading the user's progress in adhering to the treatment protocol; and
where compliance with the treatment protocol exceeds a predetermined threshold, providing an award or incentive to the user, which award may be redeemed through a mobile shopping system.
